(19) United States
(12) Patent Application Publication
(10) Pub. No.: US 2013/0039957 A1
(43) Pub. Date: Feb. 14, 2013

(54) CONTROLLED RELEASE PHARMACEUTICAL COMPOSITIONS OF BRIVARACETAM

(71) Applicants: Raghu Rami Reddy Kasu, Pune (IN); Subhasis Das, Pune (IN); Vijaya Kumar Thommandru, Pune (IN)

(72) Inventors: Raghu Rami Reddy Kasu, Pune (IN); Subhasis Das, Pune (IN); Vijaya Kumar Thommandru, Pune (IN)

(73) Assignee: LUPIN LIMITED, Mumbai (IN)

(21) Appl. No.: 13/643,922

(22) PCT Filed: Apr. 26, 2011
(86) PCT No.: PCT/IB2011/000892

§ 371 Date: Oct. 26, 2012

(30) Foreign Application Priority Data
Apr. 29, 2010 (IN) 489/KOL/2010

(51) Int. Cl. A61K 9/00; A61P 25/08; A61K 31/4015

(52) U.S. Cl. 424/400; 514/424

(57) ABSTRACT

The present invention relates to controlled release pharmaceutical compositions comprising Brivaracetam or its pharmaceutically acceptable derivatives thereof. Further disclosed is a controlled release pharmaceutical composition comprising a core and a coating surrounding the core, wherein the core comprises Brivaracetam or pharmaceutically acceptable derivative thereof and the coating comprises hydrophobic release controlling agent. The controlled release pharmaceutical composition comprises Brivaracetam or pharmaceutically acceptable derivatives thereof and hydrophobic release controlling agent, wherein said composition has dissolution of Brivaracetam at least 80% between about 7 to about 24 hours when measured in 900 ml of pH 6 phosphate buffer solution using USP apparatus type II, at 50 rpm and at 37° C. Also disclosed is a controlled release pharmaceutical composition useful for the treatment of epilepsy and treatment of symptomatic myoclonus comprises Brivaracetam or pharmaceutically acceptable derivative thereof and hydrophobic release controlling agent.

### FIELD OF THE INVENTION

The present invention relates to controlled release pharmaceutical compositions comprising Brivaracetam or its pharmaceutically acceptable derivatives thereof.

### BACKGROUND OF THE INVENTION

Epilepsy is a relatively common neurological condition affecting 0.4-1% of the world's population (45-100 million people). For the general population there are approximately 20-70 new cases per 100,000 diagnosed each year with a 3-5% lifetime probability of developing the disease.

Epilepsy is a common chronic neurological disorder characterized by recurrent unprovoked seizures. These seizures are transient signs and/or symptoms of abnormal, excessive or synchronous neuronal activity in the brain. It is classified etiologically as symptomatic or idiopathic with seizure manifestations that fall into three general categories: 1) generalized tonic-clonic, 2) absence or petit mal, and 3) complex partial. Symptomatic classification indicates that a probable cause exists and a specific course of therapy to eliminate that cause may be tried, whereas idiopathic indicates that no obvious cause can be found and may be linked to unexplained genetic factors. Of the seizure categories, most people have only one type of seizure, while about 30% have two or more types.

Currently, various drugs are available for the treatment of epilepsy or epileptic condition, and commonly referred to as anticonvulsants or antiepileptics. Example of these drugs includes carbamazepine, sodium valproate, phenytoin sodium, ethosuximide, clonazepam, diazepam, nitrazepam, primidone, phenobarbitone, gabapentin, pregabalin, progabide, vigabatrin, lamotrigine, topiramate and levetiracetam. These drugs are marketed in different dosage forms such as conventional immediate release tablets, capsules and the like or controlled release dosage forms in several geographies.

Most conventional oral drug products, such as tablets and capsules, release the active drug immediately after oral administration over a shorter period of time, such as 60 minutes or less, to attain rapid systemic drug absorption and have quick onset of pharmacodynamic effects. As immediate release drug products are absorbed into the body quickly, there will be a sharp rise in blood levels. These ‘peaks’ may be associated with side effects such as dizziness, drowsiness and lack of coordination. However, plasma drug concentration declines, according to the drug's pharmacokinetic profile and eventually it falls below the minimum effective plasma concentration (MEC), resulting in loss of therapeutic activity.

To maintain reasonably stable plasma concentrations, it is necessary to resort frequent dosing. This leads to substantial fluctuations in the plasma concentration of the drug, especially in chronic administration. Moreover, frequent dosing of immediate release drug products results into inconvenience to the patient which leads to decreased patient compliance.

To overcome disadvantage associated with immediate release drug products, a concerted effort has been devoted to the discovery of controlled release drug products. Controlled release drug products offer several advantages over immediate-release drug products of the same drug. Controlled release allows sustained therapeutic blood levels of the drug for a prolonged period of time and consistent clinical response in the patient. As in controlled release drug products, the drug input rate is constant, the blood levels do not fluctuate which leads to better patient convenience and patient compliance.

Brivaracetam is a n-propyl derivative of levetiracetam which has a molecular structure as

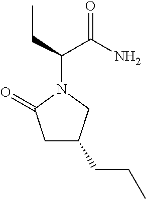

It has been studied for various potential indications, including epilepsy, neuropathic pain, and essential tremors, among others. The drug interacts selectively with specific binding sites in the brain.

In preclinical studies Brivaracetam showed affinity for synaptic vesicle protein 2A (SV₂A). Brivaracetam also has inhibitory activity at neuronal voltage-dependent sodium channels whose abnormal function is understood to contribute to electrical discharges associated with seizures.

Brivaracetam is effective in the treatment of epilepsy. Clinical trials evaluated the efficacy and safety of Brivaracetam (5, 20, 50 and 150 mg per day) in the adjunctive treatment of adult patients (16-65 years) with refractory partial onset seizures, with or without secondary generalization.

Pharmacokinetic studies shows that Brivaracetam is water soluble drug (700 mg/ml) with a half life of 7.6±1.7 hours and Cₘₐₓ of 1.5 hours post dose (Maria et al, Pharmacokinetics and Metabolism of ¹⁴C-Brivaracetam, A Novel SV₂A Ligand in Healthy Subjects, American Society for Pharmacology and Experimental Therapeutics: October 2007).

Brivaracetam has short half-life and high water solubility. These characteristics are ideal to develop controlled release pharmaceutical compositions which invariably will offer various advantages over conventional immediate release drug products such as constant therapeutic plasma concentration of Brivaracetam over prolonged periods, reduced number of doses per day or week, reduced adverse effects and improved patient compliance and convenience.

WO 2009/144286 discloses a pharmaceutical composition in the form of a tablet comprising Brivaracetam and, as excipient within the core of the tablet, 5% to 80% per weight of at least one hydrophilic matrix agent, with respect to the total weight of the core of the tablet.

Hydrophilic polymer matrix systems are widely used to develop controlled pharmaceutical compositions because of their flexibility to obtain a desirable drug release profile, cost-effectiveness, and broad regulatory acceptance. However, the drug release for extended duration, particularly for highly water-soluble drugs, using a hydrophilic matrix system is restricted due to rapid diffusion of the dissolved drug through the hydrophilic gel network. Faster release of the drug from the hydrophilic matrix is probably due to faster dissolution of the highly water-soluble drug from the core and its diffusion out of the matrix forming the pores for entry of solvent molecules.

Hydrophobic controlled release agents are the most suitable controlled release agents for the development of controlled release pharmaceutical compositions of Brivaracetam with high water solubility. This is attributed to the decreased penetration of the solvent molecules in the presence of a hydrophobic polymer leading to decreased diffusion of the drug from the pharmaceutical composition.

Thus the present invention provides a controlled release pharmaceutical composition comprising Brivaracetam or pharmaceutically acceptable derivative thereof and hydrophobic release controlling agent.

### OBJECT OF THE INVENTION

Therefore one embodiment provides controlled release pharmaceutical compositions comprising Brivaracetam or pharmaceutically acceptable derivatives and hydrophobic release controlling agent.

Further embodiment provides controlled-release pharmaceutical compositions comprising Brivaracetam or pharmaceutically acceptable derivatives thereof and hydrophobic release-controlling agent adapted to release Brivaracetam over a predetermined time period, at least for about 24 hours. A suitable dissolution test is where the measurement is carried out in 900 ml of pH 6 phosphate buffer solution; using USP apparatus type II, at 50 rpm and at 37° C. or variations of this as well known to one who is skilled in the art.

Another embodiment proposes controlled release pharmaceutical compositions of Brivaracetam or pharmaceutically acceptable derivatives thereof wherein the complete dissolution time that is the time for release of at least 80% of the total amount of the drug is between about 7 to about 24 hours, preferably between about 8 to about 20 hours when dissolution is carried out in 900 ml of pH 6 phosphate buffer solution, using USP apparatus type II, at 50 rpm and at 37° C. or variations of this as well known to one who is skilled in the art.

Further embodiment proposes controlled release pharmaceutical compositions of Brivaracetam or pharmaceutically acceptable derivative thereof wherein pharmaceutical composition releases at least about 40% of the drug in about 1.5 hours when dissolution is carried out in 900 ml of pH 6 phosphate buffer solution, using USP apparatus type II, at 50 rpm and at 37° C. or variations of this as well known to one who is skilled in the art.

Still further embodiment provides controlled release compositions comprising Brivaracetam or pharmaceutically acceptable derivatives and hydrophobic release controlling agent for the treatment of epilepsy and treatment of symptomatic myoclonus.

### BRIEF DESCRIPTION OF THE DRAWINGS

FIG. 1a shows a release profile of controlled release pharmaceutical composition of Brivaracetam of Example 1, in 900 ml pH 6 phosphate buffer solution, USP apparatus Type II, at 37° C., 50 rpm.

FIG. 1b shows a release profile of controlled release pharmaceutical composition of Brivaracetam of Example 1, in 900 ml water, USP apparatus Type II, at 37° C., 50 rpm.

FIG. 2 shows a release profile of controlled release pharmaceutical composition of Brivaracetam of example 6, in 900 ml pH 6 phosphate buffer solution, USP apparatus Type II, at 37° C., 50 rpm.

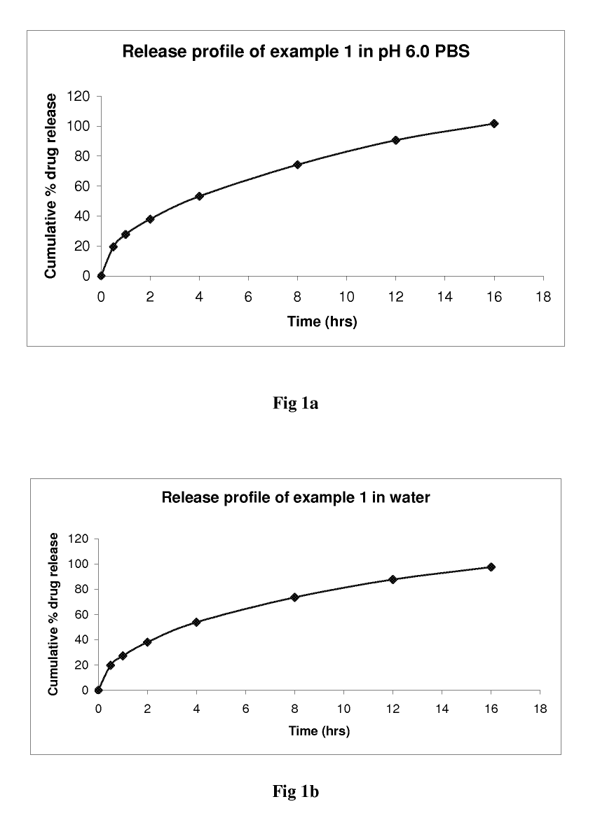

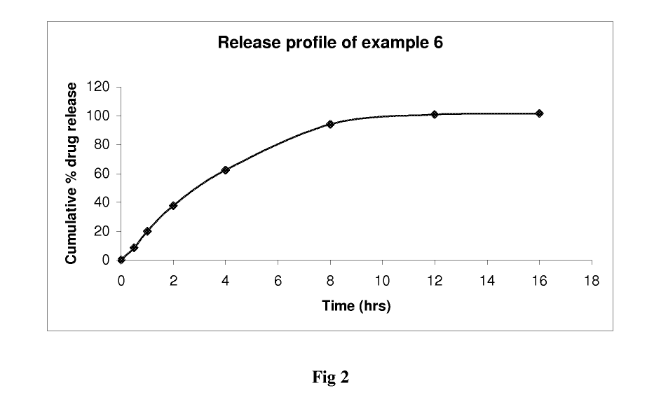

### DETAILED DESCRIPTION OF THE INVENTION

One embodiment provides controlled release pharmaceutical compositions comprising Brivaracetam or pharmaceutically acceptable derivatives thereof, which provides at least about 80% of the drug is released in 24 hrs or the pharmaceutical composition of the present invention can be suitably designed to provide controlled release pharmaceutical compositions that control release of the active over prolonged periods of time, at least for, 20 hours after oral administration.

As used herein “Brivaracetam” also encompasses pharmaceutically acceptable derivatives of Brivaracetam including enantiomers of Brivaracetam and mixture thereof, pharmaceutically acceptable salts, esters, prodrugs, analogues and active metabolites of Brivaracetam and their pharmaceutically acceptable salts, unless otherwise noted.

The amount of Brivaracetam or pharmaceutically acceptable derivatives may range from about 2.5 to about 500 mg.

The term “controlled release pharmaceutical compositions” herein refers to any composition or dosage form which comprises an active drug and which is formulated to provide a longer duration of pharmacological response after administration of the dosage form than is ordinarily experienced after administration of a corresponding immediate release composition comprising the same drug in the same amount. Controlled release pharmaceutical compositions include, inter alia, those compositions described elsewhere as “extended release”, “sustained release”, “prolonged release”, “programmed release”, “time release” and/or “rate controlled” compositions or dosage forms.

The controlled release pharmaceutical compositions are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, glidants, dissolution enhancing agents and rate controlling agents.

The rate-controlling agent(s) used in admixture with the active ingredient or in coating comprises hydrophobic release controlling agents.

The hydrophobic release controlling agent(s) are selected from but are not limited to polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, or hydrogenated vegetable oils.

The controlled release pharmaceutical compositions comprise 2 to 70% per weight of hydrophobic release controlling agent with respect to the total weight of the composition, preferably, 4 to 65% per weight of hydrophobic release controlling agent; more preferably 5 to 50% per weight of hydrophobic release controlling agent; and most preferably 6 to 40% per weight of hydrophobic release controlling agent with respect to the total weight of the tablet.

Diluents may be, for example, any pharmaceutically acceptable, non-toxic diluents, for example lactose, dextrose, sucrose, maltose, microcrystalline cellulose, starch, calcium hydrogen phosphate, mannitol and the like.

Binders may be, for example, starch, sugars, gums, low molecular weight hydroxypropyl methylcellulose, hydroxypropyl cellulose, hydroxyethyl cellulose or the like.

Lubricants may be, for example, talc, magnesium stearate, calcium stearate, stearic acid, sodium stearyl fumarate, sodium benzoate or the like.

Glidants may be, for example, colloidal silicon dioxide, talc or the like.

The term “controlled release pharmaceutical compositions” includes a pharmaceutical composition that encompasses one or more individual units. The individual units may be in form of granules, pellets, minitablets or beads. Granules, pellets, minitablets or beads of the present invention can be filled into a capsule or can be compressed into a tablet. The compositions of the invention can be further coated with suitable nonfunctional or functional coating.

The Examples below are representation only and should not be construed to limit the scope of the invention:

### Example 1

| Sr No | Ingredients | Mg/Tab |
|---|---|---|
| 1 | Brivaracetam | 50.00 |
| 2 | Lactose anhydrous | 150.00 |
| 3 | Dibasic calcium phosphate anhydrous | 33.50 |
| 4 | Colloidal silicon dioxide | 7.75 |
| 5 | Hydrogenated vegetable oil | 100.00 |
| 6 | Talc | 5.25 |
| 7 | Magnesium Stearate | 3.50 |
| 8 | Core Tablet Weight | 350 |

Seal Coating

| | | |
|---|---|---|
| 9 | Hydroxypropyl methylcellulose (HPMC E5) | 7.778 |
| 10 | Polyethylene glycol | 0.778 |
| 11 | Talc | 0.778 |
| 12 | Titanium dioxide | 1.167 |
| 13 | Isopropyl alcohol | q.s |
| 14 | Methylene chloride | q.s |
| 15 | Coated Tablet Weight | 360.50 |
| 16 | Opadry AMB | 10.82 |
| | Final Tablet Weight | 371.32 |

### Brief Manufacturing Procedure:

1) Sift Brivaracetam, Lactose anhydrous, Dibasic calcium phosphate and Colloidal silicon dioxide through #20 mesh and mix in a suitable mixer.
2) Melt hydrogenated vegetable oil (sterotex Type A) at 60 to 70° C. until it melts completely. Add the material of step 01 and granulate the same to get uniform granules.
3) Cool the granules obtained from step 02 under room temp to congeal.
4) Pass the dried granules of step 03 through #20 mesh.
5) Blend the step 4 granules with Lactose, Dibasic calcium phosphate, Colloidal silicon dioxide, talc and lubricate it with magnesium stearate and compress the lubricated blend into tablets using suitable tooling.
6) Coat the step 5 tablets with seal coat and further coat with Opadry AMB coat to give desired build up.

### Example 2

| Sr No | Ingredients | % w/w |
|---|---|---|
| 1 | Brivaracetam | 5-17.5 |
| 2 | Lactose anhydrous | 20-60 |
| 3 | Dibasic calcium phosphate anhydrous | 10-50 |
| 4 | Colloidal silicon dioxide NF | 0.5-5 |
| 5 | Hydrogenated vegetable oil | 10-60 |
| 6 | Talc | 0.5 5 |
| 7 | Magnesium Stearate | 0.5-3 |
| 8 | Seal coat | 1-3 |
| 9 | AMB (Air Moisture Barrier) coat | 1-3 |

### Brief Manufacturing Procedure:

1) Sift Brivaracetam, Dibasic calcium phosphate, Lactose anhydrous and Colloidal silicon dioxide through #20 mesh and mix in a suitable mixer.
2) Melt hydrogenated vegetable oil (sterotex Type A) at 60 to 70° C. until it melts completely. Add the material of step 1 and granulate the same to get uniform granules.
3) Cool the granules obtained from step 2 under room temp to congeal.
4) Pass the dried granules of step 3 through #20 mesh.
5) Granulate step 4 blend with dibasic calcium phosphate, colloidal silicon dioxide, talc and lubricate above blend with magnesium stearate and compress lubricated blend into tablets using suitable tooling.
6) Coat above compressed tablets with HPMC (Hydroxypropyl methyl cellulose) E1, PEG 6000, Talc and Titanium dioxide up to desired build up.
7) Coat above coated tablets with Opadry AMB white to give a 3% w/w build up.

### Example-3

| Sr No | Ingredients | % w/w |
|---|---|---|
| 1 | Brivaracetam | 5-17.5 |
| 2 | Lactose anhydrous | 25-65 |
| 3 | Dibasic calcium phosphate anhydrous | 5-20 |
| 4 | Colloidal silicon dioxide | 0.5-5 |
| 5 | Hydrogenated castor oil | 10-60 |
| 6 | Talc | 0.5-5 |
| 7 | Magnesium Stearate | 0.5-3.5 |
| 8 | AMB coat | 1-3 |

### Brief Manufacturing Procedure:

1) Sift Brivaracetam, Lactose anhydrous and Colloidal silicon dioxide through #20 mesh and mix in a suitable mixer.
2) Melt Hydrogenated Castor Oil at 60 to 70° C. until it melts completely. Add the material of step 1 and granulate the same to get uniform granules.
3) Cool the granules obtained from step 02 under room temp to congeal.
4) Pass the dried granules of step 03 through #20 mesh.
5) Blend the granules of step 4 with Lactose, Dibasic calcium phosphate, Colloidal silicon dioxide, talc and lubricate with magnesium stearate and compress lubricated blend into tablets using suitable tooling.
6) Coat above coated tablets with Opadry AMB white to give a 3% w/w build up.

### Example 4

| | | |
|---|---|---|
| S. No. | Ingredients | % w/w |

Intra granular

| | | |
|---|---|---|
| 1 | Brivaracetam | 15-25 |
| 2 | Lactose monohydrate | 30-70 |
| 3 | Pre-gelatinised Starch | 5-25 |

Extra granular

| | | |
|---|---|---|
| 4 | Magnesium stearate | 0.5-3 |

Ethyl cellulose - HPMC coating:

| | | |
|---|---|---|
| 7 | Ethyl cellulose 10 CPS | 0.5-20 |
| 8 | HPMC 15 cps | 0.05-10 |
| 9 | Dibutyl Sebacate | 0.05-4 |
| 10 | Isopropyl alcohol | q.s |
| 11 | Dichloro methane | q.s |
| 12 | Opadry white AMB | 1-4 |
| 14 | Purified water | q.s |

### Brief Manufacturing Procedure:

1) Sift Brivaracetam, lactose and pregelatinised starch through #30 ASTM mesh and granulate water and dry the wet mass at 50° C.-55° C.
2) Pass dried granules through #25 ASTM mesh and lubricate with Magnesium stearate (previously passed through #40 mesh) for 5 min. and compress the tablets using suitable punches.
3) Coated the above tablets with the solution of Ethyl cellulose and HPMC to a weight gain of 10-15% w/w
4) Coat the step 3 tablets with Opadry white AMB to give a 3% w/w build up.

### Example 5

| | | |
|---|---|---|
| S. No. | Ingredients | % w/w |

Intra granular

| | | |
|---|---|---|
| 1 | Brivaracetam | 15-25 |
| 2 | Lactose monohydrate | 30-70 |
| 3 | Pre-gelatinised Starch | 5-25 |

Extra granular

| | | |
|---|---|---|
| 4 | Magnesium stearate | 0.5-3 |
| | Core tablets weight | 60-90 |
| 7 | Ethyl cellulose 10 CPS | 0.5-3 |
| 8 | HPMC 15 cps | 0.05-10 |
| 9 | Dibutyl Sebacate | 0.05-4 |
| 10 | Isopropyl alcohol | q.s |
| 11 | Dichloro methane | q.s |
| | Weight of Ethyl Cellulose Coated tablets | 80-100 |
| 13 | Opadry white AMB | 2.91 |
| 14 | Purified water | q.s |
| | Weight of Coated tablets | 100 |

### Brief Manufacturing Procedure:

1) Sift Brivaracetam, lactose and pregelatinised starch through #30 ASTM mesh and granulate water and dry the wet mass at 50° C.-55° C.
2) Pass dried granules through #30 ASTM mesh and lubricate with Magnesium stearate (previously passed through #40 mesh) for 5 minute and compress the tablets using suitable punches into mini tablets.
3) Coat the above tablets with the solution of Ethyl cellulose and HPMC to a weight gain of 10-15% w/w
4) Coat the step 3 tablets with Opadry white AMB to give a 3% w/w build up.
5) Fill the coated tablets of step 4 into capsules of suitable size.

### Example 6

| | | |
|---|---|---|
| S. No. | Ingredients | % w/w |

Intra granular

| | | |
|---|---|---|
| 1 | Brivaracetam | 15-25 |
| 2 | Lactose monohydrate | 30-70 |
| 3 | Pre-gelatinised Starch | 5-25 |
| 4 | Magnesium stearate | 0.5-3 |

Ethyl cellulose - Povidone coating:

| | | |
|---|---|---|
| 5 | Ethyl cellulose 10 CPS | 0.5-20 |
| 6 | Povidone K 90 | 0.05-10 |
| 7 | Dibutyl Sebacate | 0.05-4 |
| 8 | Isopropyl alcohol | q.s |
| 9 | Dichloro methane | q.s |
| 10 | Weight of Ethyl cellulose Coated tablets | 90-100 |
| 11 | Opadry white AMB | 1-4 |
| 12 | Purified water | q.s |
| | Weight of Coated tablets | 100 |

### Brief Manufacturing Procedure:

1) Sift Brivaracetam, lactose and pregelatinised starch through #30 ASTM mesh and granulate water and dry the wet mass at 50° C.-55° C.
2) Pass dried granules through #25 ASTM mesh and lubricate with Magnesium stearate (previously passed through #40 mesh) for 5 min. and compress the tablets using suitable punches.
3) Coat the above tablets with the solution of Ethyl cellulose and Povidone to a weight gain of 10-15% w/w.
4) Coat the step 3 tablets with Opadry white AMB to give 3% w/w build up.

### Example 7

| | | |
|---|---|---|
| S. No | Ingredients | % w/w |

Intra granular

| | | |
|---|---|---|
| 1 | Brivaracetam | 5-20 |
| 2 | Lactose anhydrous | 20-65 |
| 4 | Colloidal silicon dioxide | 0.5-5 |
| 5 | Hydrogenated vegetable oil | 10-60 |
| 6 | Dibasic calcium phosphate | 5-20 |
| 7 | Talc | 0.5-5 |
| 8 | Magnesium stearate | 0.5-3 |

### Brief Manufacturing Procedure:

1) Sift Brivaracetam, Lactose anhydrous and Colloidal silicon dioxide through #20 mesh and mix in a suitable mixer.
2) Melt hydrogenated vegetable oil (sterotex Type A) at 60 to 70° C. until it melts completely. Add the material of step 01 and granulate the same to get uniform granules.
3) Cool the granules obtained from step 02 under room temp to congeal.
4) Pass the dried granules of step 03 through #20 mesh.
5) Blend the step 4 granules with Lactose, Dibasic calcium phosphate, Colloidal silicon dioxide, talc and lubricate it with magnesium stearate and compress the lubricated blend into tablets using suitable tooling.
6) Fill the tablets of step 5 in a capsule of suitable size.

### Example 8

| Sr No | Ingredients | % w/w |
|---|---|---|
| 1 | Brivaracetam | 15-30 |
| 2 | Sugar pellets | 50-75 |
| 3 | HPMC E3 | 2.5-7.5 |
| 4 | Talc | 0.5-5 5 |
| 5 | Ethyl cellulose 10 CPS | 2.5-15 |
| 6 | HPMC 15 cps | 0.5-10 |
| 7 | Dibutyl Sebacate | 0.05-5 |

### Brief Manufacturing Procedure:

1) Dissolve Brivaracetam and HPMC in sufficient quantity of water and load this solution on the sugar pellets using Fluidized Bed Processor.
2) Coat above drug loaded pellets using Ethyl cellulose and HPMC solution in (IPA: DCM (1:1)) to give and required wt build up of 0-25%.
3) Fill the above-coated pellets into capsules or Blended with suitable compression-aiding material and compressed into tablets.

1. A controlled release pharmaceutical composition comprising Brivaracetam or pharmaceutically acceptable derivative thereof and hydrophobic release controlling agent.

2. The controlled release pharmaceutical composition according to claim 1, wherein the hydrophobic release controlling agent is selected from ethyl cellulose, polyvinyl acetate dispersion, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, hydrogenated vegetable oil or glyceryl behenate.

3. The controlled release pharmaceutical composition according to claim 1, wherein the hydrophobic release controlling agent is present in admixture with Brivaracetam or pharmaceutically acceptable derivative thereof or in the coating.

4. A controlled release pharmaceutical composition comprising a core and a coating surrounding the core, wherein the core comprises Brivaracetam or pharmaceutically acceptable derivative thereof and the coating comprises hydrophobic release controlling agent.

5. The controlled release pharmaceutical composition according to claim 4, wherein the hydrophobic release controlling agent is selected from ethyl cellulose, polyvinyl acetate dispersion, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate) or poly (octadecyl acrylate).

6. The controlled release pharmaceutical composition according to claim 5, wherein the hydrophobic release controlling agent is ethyl cellulose.

7. A controlled release pharmaceutical composition comprising Brivaracetam or pharmaceutically acceptable derivatives thereof and hydrophobic release controlling agent, wherein said composition has dissolution of Brivaracetam at least 80% between about 7 to about 24 hours when measured in 900 ml of pH 6 phosphate buffer solution using USP apparatus type II, at 50 rpm and at 37° C.

8. A controlled release pharmaceutical composition of claim 7, wherein the composition has dissolution of Brivaracetam at least 80% between about 8 to about 20 hours when measured in 900 ml of pH 6 phosphate buffer solution using USP apparatus type II, at 50 rpm and at 37° C.

9. A controlled release pharmaceutical composition useful for the treatment of epilepsy and treatment of symptomatic myoclonus comprises Brivaracetam or pharmaceutically acceptable derivative thereof and hydrophobic release controlling agent.
